(19) United States
(12) Patent Application Publication
(10) Pub. No.: US 2026/0158239 A1
(43) Pub. Date: Jun. 11, 2026

(54) TAMPER-DETERRENT DEVICES FOR MEDICAL CATHETERS

(71) Applicant: THE UNIVERSITY OF NORTH CAROLINA AT CHAPEL HILL, Chapel Hill, NC (US)

(72) Inventors: Nicole WILEY, Pittsboro, NC (US); Dana MABRY, Pittsboro, NC (US); Nabil KHAN, Chapel Hill, NC (US); Robert LAMPMAN, Apex, NC (US)

(21) Appl. No.: 19/126,184

(22) PCT Filed: Nov. 2, 2023
(86) PCT No.: PCT/US2023/036700

§ 371 Date: Apr. 30, 2025

Related U.S. Application Data
(60) Provisional application No. 63/382,383, filed on Nov. 4, 2022.

(51) Int. Cl. A61M 25/00; E05B 47/00; E05B 51/00

(52) U.S. Cl. CPC A61M 25/0043; E05B 47/0045; E05B 51/005; A61M 2205/0266; A61M 2205/0272; A61M 2205/276

(57) ABSTRACT

A tamper-deterrent device for medical catheters includes a housing and a locking mechanism. The housing includes first and second portions movably connected to one another and movable relative to one another between open and closed positions. The first and second housing portions are configured to enclose a connection, such as access claves, between at least two catheters when in the closed position. The locking mechanism is configured to secure the first and second housing portions in the closed position and includes a magnetically-operated locking pin that is configured to be moved by a contactless magnetic key from a locking position to a release position. The locking mechanism may include a locking pin configured to be moved from a locking position to a release position by a shape memory alloy (SMA) element when electric current is applied to the SMA element.

### RELATED APPLICATION

This application claims the benefit of and priority to U.S. Provisional Patent Application No. 63/382,383 filed Nov. 4, 2022, the disclosure of which is incorporated herein by reference as if set forth in its entirety.

### GOVERNMENT SUPPORT

This invention was made with Government support under Grant Number TR002489 awarded by the National Institutes of Health. The United States Government has certain rights to this invention.

### FIELD

The present inventive concept relates generally to medical catheters and, more particularly, to devices for preventing unauthorized access to medical catheters that are attached to patients.

### BACKGROUND

Patients with a history of substance abuse that are admitted to the hospital are at increased risk of tampering with their peripherally inserted central catheter (PICC) or central venous catheter (CVC) line, using it as a route of administration for illicit drugs. Improper patient line access may lead to central line acquired blood stream infections (CLABSI) which may extend the patient's length of stay in the hospital and may increase hospital infection rates. Conventional devices for preventing such unauthorized access are only tamper-evident and may be easily removed/bypassed by both a clinician and a patient. These devices must be replaced every time a line is accessed, either by a clinician or improperly by a patient, which makes them cost prohibitive since the lines typically are accessed multiple times a day by a clinician. In addition, these conventional devices are designed for when a catheter line is not being accessed, so improper access by the patient during continuous infusions may still occur.

### SUMMARY

The present inventive concept relates to a two-part device consisting of a tamper-deterrent housing with a reusable magnetic or electronic locking mechanism. The disposable housing encases PICC/CVC access claves during and after infusion and the locking mechanism prevents the housing from being opened without the correct activation key. The housing can be discarded between patients or if it becomes soiled during use with a single patient. The locking mechanism can be sanitized and reused between patients. Embodiments of the present inventive concept may be used in settings other than hospitals, such as skilled nursing facilities and home health settings.

According to some embodiments of the present inventive concept, a tamper-deterrent device for medical catheters includes a housing and a locking mechanism. The housing includes first and second portions movably connected to one another (e.g., connected by one or more hinges, etc.) and are movable relative to one another between open and closed positions. The first and second housing portions are configured to enclose a connection, such as access claves, between at least two catheters when in the closed position. The housing includes openings for the at least two catheters to extend therethrough, for example, at opposite ends of the housing.

The locking mechanism is configured to secure the first and second housing portions in the closed position and includes a magnetically-operated locking pin that is configured to be moved by a contactless magnetic activation key from a locking position to a release position. The locking mechanism also includes a spring configured to urge the locking pin to the locking position. The locking mechanism is secured to one of the first and second housing portions, and the locking pin is configured to engage a portion of the other one of the first and second housing portions when in the locking position. In some embodiments, the locking mechanism is removably secured to the one of the first and second housing portions. In some embodiments, the locking mechanism is located within the housing.

According to other embodiments of the present inventive concept, the locking mechanism includes a locking pin configured to be moved from a locking position to a release position by a shape memory alloy (SMA) element when electric current is applied to the SMA element. The locking mechanism also includes a spring that is configured to urge the locking pin to the locking position. The device includes a power source that is configured to provide the electric current to the SMA element in response to activation by a contactless key. In some embodiments, the power source is located within the housing. In some embodiments, the SMA element is a wire. The locking mechanism is secured to one of the first and second housing portions, and the locking pin is configured to engage a portion of the other one of the first and second housing portions when in the locking position. In some embodiments, the locking mechanism is removably secured to the one of the first and second housing portions. In some embodiments, the locking mechanism is located within the housing.

According to other embodiments of the present inventive concept, the locking mechanism includes a locking pin and a shape memory alloy (SMA) spring. The SMA spring is configured to move the locking pin from the locking position to a release position when electric current is applied thereto. The device includes a power source that is configured to provide the electric current to the SMA spring in response to activation by a contactless key. In some embodiments, the power source is located within the housing. The locking mechanism is secured to one of the first and second housing portions, and the locking pin is configured to engage a portion of the other one of the first and second housing portions when in the locking position. In some embodiments, the locking mechanism is removably secured to the one of the first and second housing portions. In some embodiments, the locking mechanism is located within the housing.

It is noted that aspects of the present inventive concept described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present inventive concept are explained in detail below.

### BRIEF DESCRIPTION OF THE DRAWINGS

The accompanying drawings, which form a part of the specification, illustrate various embodiments of the present inventive concept. The drawings and description together serve to fully explain embodiments of the present inventive concept.

FIG. 1 illustrates a housing, a locking mechanism, and an activation key for a tamper-deterrent device for medical catheters, according to some embodiments of the present inventive concept.

FIG. 2A is a perspective view of a tamper-deterrent device for medical catheters, according to some embodiments of the present inventive concept, and with the housing in an open position illustrating the locking mechanism.

FIG. 2B is a perspective view of the device of FIG. 2A with the housing in a closed position enclosing one or more connections of one or more catheters.

FIG. 3 is a plan view of the first and second portions of the housing of the device of FIG. 1 and illustrating access claves of a pair of catheters positioned within the housing and also illustrating the locking mechanism.

FIG. 4A is a plan view of the housing portion of FIG. 3 that contains the locking mechanism and also illustrating the locking pin receiving portion from the other housing portion in position for receiving the locking pin therewithin.

FIG. 4B illustrates the housing portion of FIG. 4A with the housing for the locking mechanism removed and the locking pin receiving portion from the other housing portion removed to illustrate the magnetic locking pin and spring.

FIG. 5A is a cutaway perspective view of the two housing portions of FIG. 3 in a closed position.

FIG. 5B is a cross sectional view of the device of FIG. 5A taken along line 5B-5B.

FIG. 6 is a plan view of the device of FIG. 3 with the housing portions in the closed position and showing movement of the magnetic activation key to move the locking pin from a locking position to a release position.

FIG. 7 is a perspective view of a tamper-deterrent device for medical catheters, according to other embodiments of the present inventive concept, and with the housing in an open position.

FIG. 8 is a plan view of the first and second portions of the housing of the device of FIG. 7 and illustrating access claves of a pair of catheters positioned within the housing and also illustrating the locking mechanism.

FIG. 9A is a plan view of the housing portion of FIG. 8 that contains the locking mechanism and also illustrating the locking pin receiving portion from the other housing portion in position for receiving the locking pin therewithin.

FIG. 9B illustrates the housing portion of FIG. 9A with the housing for the locking mechanism removed and the locking pin receiving portion from the other housing portion removed to illustrate the locking pin and spring. Also illustrated is the SMA element that is configured to move the locking pin from a locking position to a release position when electric current is applied to the SMA element.

FIG. 10 illustrates the housing portion of FIG. 9A with example locations for a power source for providing electric current to the SMA element.

FIG. 11A is a plan view of the device of FIG. 8 with the housing portions in the closed position and showing a location of the activation key required to activate the power source in the lower portion of FIG. 10.

FIG. 11B is a plan view of the device of FIG. 8 with the housing portions in the closed position and showing a location of the activation key required to activate the power source in the upper portion of FIG. 10.

FIG. 12 is a plan view of an SMA spring that may be utilized within a tamper-deterrent device for medical catheters, according to some embodiments of the present inventive concept.

FIG. 13 is a plan view of a tamper-deterrent device for medical catheters with first and second housing portions in an open position illustrating access claves of a pair of catheters positioned within the housing and also illustrating the locking mechanism that contains the SMA spring of FIG. 12.

FIG. 14A is a plan view of the housing portion of FIG. 13 containing the locking mechanism and also illustrating the locking pin receiving portion from the other housing portion in position for receiving the locking pin therewithin.

FIG. 14B illustrates the housing portion of FIG. 14A with the housing for the locking mechanism removed and the locking pin receiving portion from the other housing portion removed to illustrate the locking pin and SMA spring.

FIG. 15 illustrates the housing portion of FIG. 14A with example locations for a power source for providing electrical current to the SMA spring.

FIG. 16A is a plan view of the device of FIG. 13 with the housing portions in the closed position and showing a location of the activation key required to activate the power source in the lower portion of FIG. 15.

FIG. 16B is a plan view of the device of FIG. 13 with the housing portions in the closed position and showing a location of the activation key required to activate the power source in the upper portion of FIG. 15.

### DETAILED DESCRIPTION

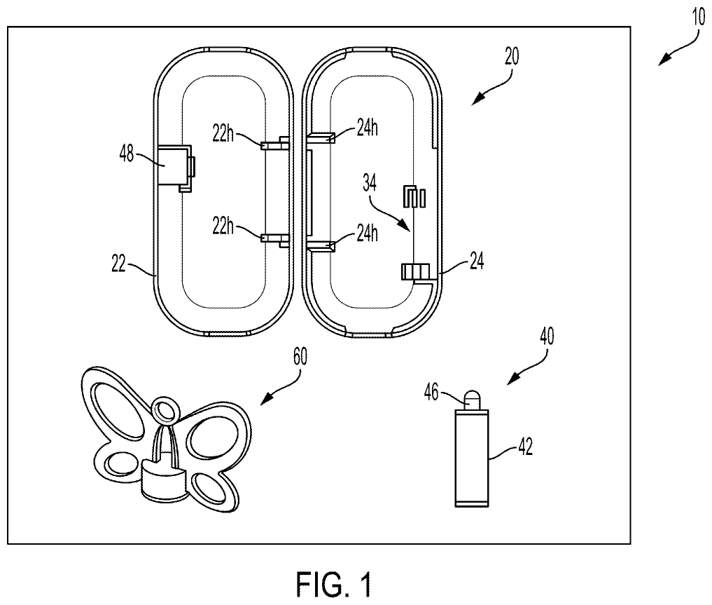

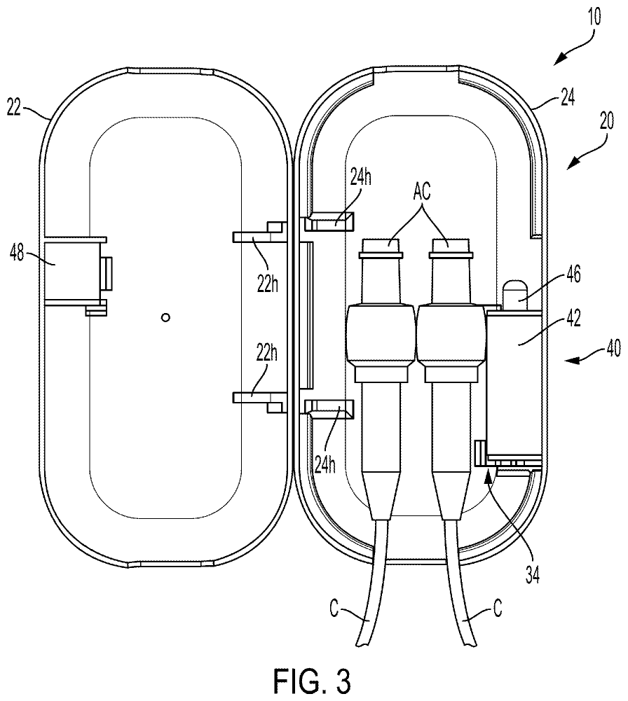

Referring initially to FIGS. 1, 2A-2B, 3, 4A-4B, 5A-5B and 6, a device 10 for enclosing connections AC (e.g., PICC/CVC access claves) between medical catheters C, according to some embodiments of the present inventive concept, will be described. The illustrated device 10 includes a tamper-deterrent housing 20, a locking mechanism 40, and an activation key 60 that is configured to operate the locking mechanism 40. The housing 20 is configured to encase the PICC/CVC access claves AC during and after infusion and the locking mechanism 40 prevents the housing 20 from being opened without the activation key 60.

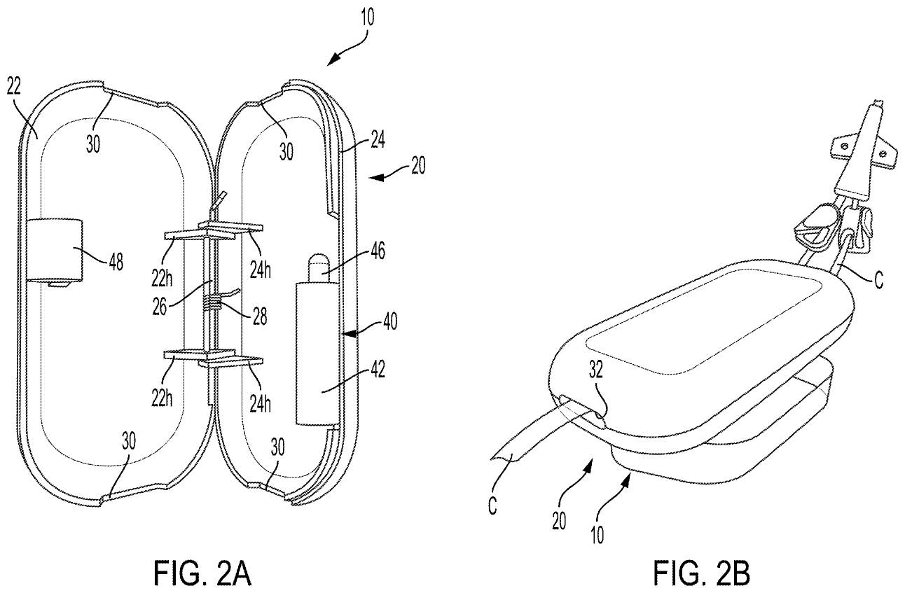

The illustrated housing 20 includes first and second portions 22, 24 configured to be movably connected to one another. In the illustrated embodiment, each housing portion 22, 24 includes respective hinge portions 22h, 24h that are configured to receive a hinge pin 26 (FIG. 2A) therethrough such that the housing portions 22, 24 are movable between open and closed positions. In the illustrated embodiment, a spring 28 is attached to the hinge pin 26 and is configured to urge the housing portions 22, 24 to an open position when the locking pin 46 is moved to a release position, as will be described below. However, the housing portions 22, 24 can be movably connected together in various ways. Embodiments of the present inventive concept are not limited to the illustrated hinged configuration. Moreover, the housing 20 can be manufactured as a single part with a hinge incorporated between the two portions 22, 24. Embodiments of the present invention do not require the two housing portions 22, 24 to be manufactured separately. In some embodiments, the hinge may be configured to urge the two portions 22, 24 open. In some embodiments, a spring-like feature can be added to urge the housing portions 22, 24 to an open position when the locking pin 46 is moved to a release position.

Each housing portion 22, 24 includes a recessed portion 30 in each end, as illustrated in FIG. 2A. When the housing portions 22, 24 are in the closed position, these recessed portions 30 create respective openings 32 in opposite ends of the housing 20 through which catheters C can extend, as illustrated in FIG. 2B. However, embodiments of the present inventive concept are not limited to the illustrated size, configuration, number or location of the openings in the housing 20. The housing portions 22, 24 may be formed from various lightweight materials, such as polymeric materials, and are intended to be discarded after use by a single patient or if soiled during use by a patient.

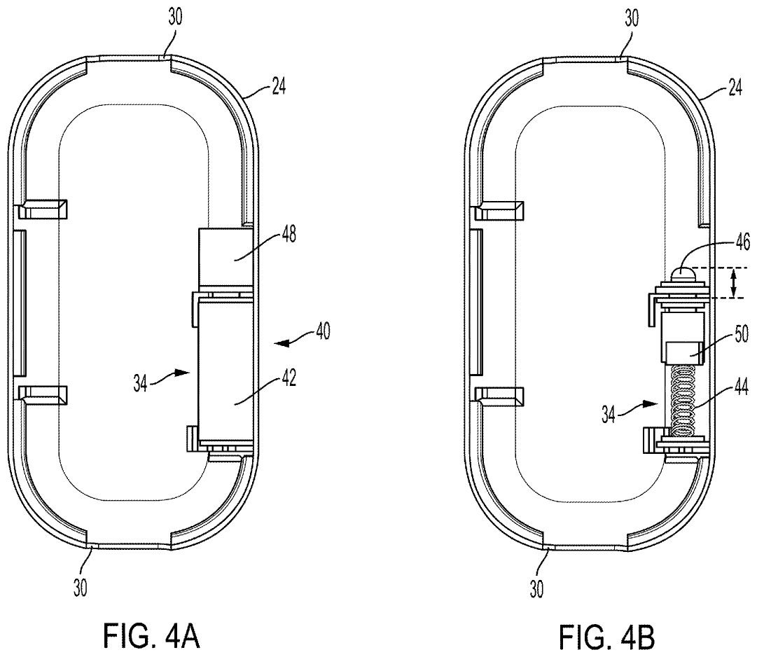

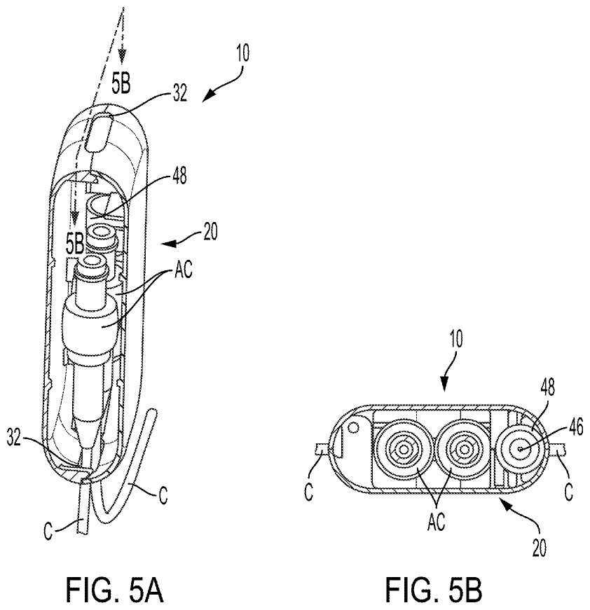

The locking mechanism 40 is configured to secure the first and second housing portions 22, 24 in the closed position. In some embodiments, the locking mechanism 40 is removably secured to the housing 20 and is intended to be removed therefrom, sterilized and reused in other housings 20 multiple times. In the illustrated embodiment, housing portion 24 includes a cradle or receptacle 34 within which the locking mechanism can be removably secured, such as via a snap fit, etc. The illustrated locking mechanism 40 includes a housing 42 containing a spring 44 and a locking pin 46, as illustrated in FIGS. 4A-4B. The spring 44 is configured to urge the locking pin 46 outwardly from the housing 42 to an extended locking position. When the first and second housing portions 22, 24 are closed and the locking pin is in the extended locking position, the locking pin 46 is inserted within a receiving portion 48 of the first housing portion 22, as illustrated in FIGS. 4A and 5B.

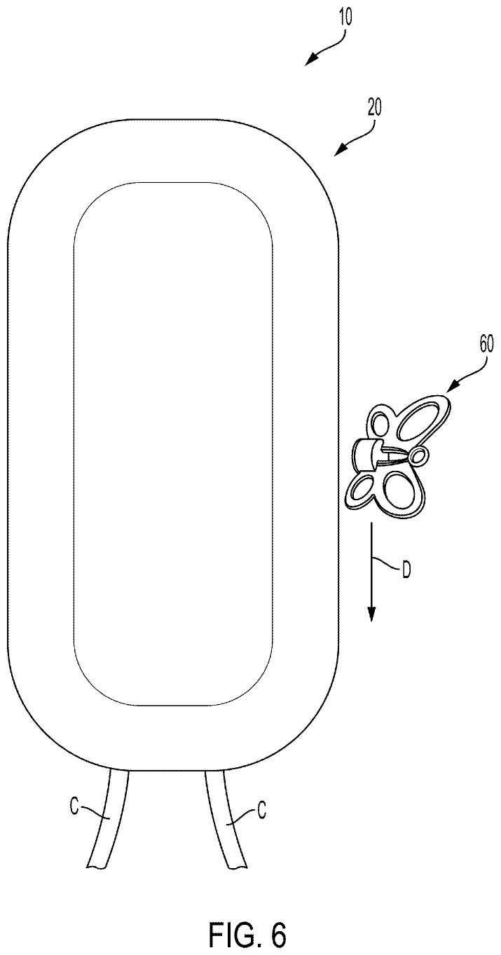

The locking pin 46 is magnetically operated and configured to be moved by a contactless magnetic key (60, FIGS. 1 and 6) from the locking position to a release position, such that the first and second housing portions 22, 24 can be moved to an open position. In the illustrated embodiment, one or more magnets 50 are attached to the locking pin 46, as shown in FIG. 4B. In other embodiments, the locking pin itself may be magnetic.

In operation, when the housing portions 22, 24 are moved to the closed position to encase the connection(s) AC of one or more catheters C therewithin, the activation key 60 is placed close to the housing 20 (and need not make contact with the housing 20) and moved in the direction D shown in FIG. 6 to move the locking pin 46 to a retracted release position such that the housing portions 22, 24 can be closed completely. The activation key 60 is then removed from the vicinity of the housing 20 and the spring 44 urges the locking pin 46 to the extended locking position so that the locking pin 46 is inserted with the receiving portion 48. Similarly, to open the housing when in the closed position and the locking pin 46 is in the extended locking position, the activation key 60 is moved in the direction D shown in FIG. 6 to move the locking pin 46 to a retracted release position, thereby allowing the housing portions 22, 24 to open. The spring 28 associated with the hinge pin 26 urges the housing portions 22, 24 open when the locking pin 46 is moved to the retracted release position.

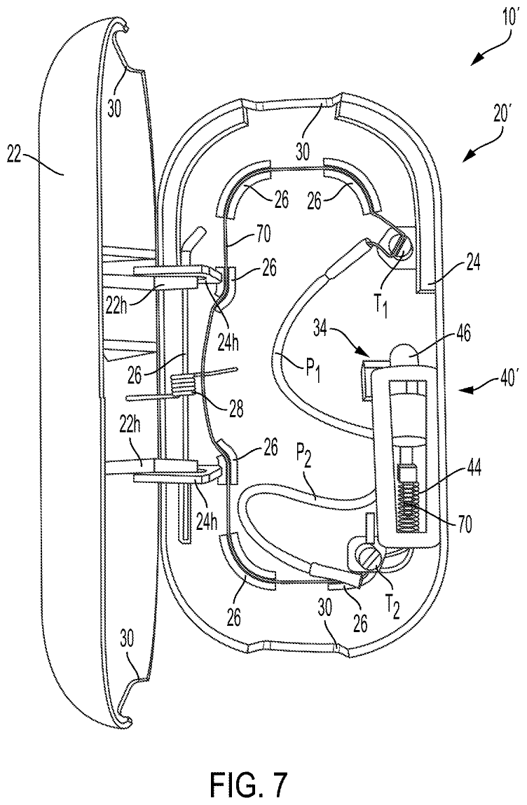

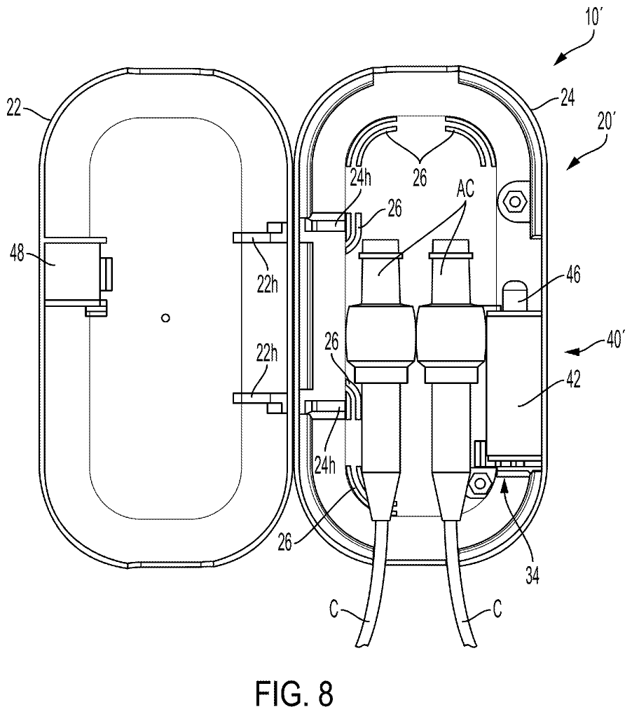

Referring to FIGS. 7, 8, 9A-9B, 10 and 11A-11B, a device 10′ for enclosing connections AC (e.g., PICC/CVC access claves) between medical catheters C, according to other embodiments of the present inventive concept will be described. Similar to the device 10 described above, the device 10′ includes a tamper-deterrent housing 20′, a locking mechanism 40′, and a contactless activation key 60 that is configured to operate the locking mechanism 40′. The housing 20′ is configured to encase the PICC/CVC access claves AC during and after infusion and the locking mechanism 40′ prevents the housing 20′ from being opened without the activation key 60.

The illustrated housing 20′ is similar to the housing 20 described above and includes first and second portions 22, 24 configured to be movably connected to one another. Each housing portion 22, 24 includes respective hinge portions 22h, 24h that are configured to receive a hinge pin 26 therethrough such that the housing portions 22, 24 are movable between open and closed positions. A spring 28 is attached to the hinge pin 26 and is configured to urge the housing portions 22, 24 to an open position when the locking pin 46 is moved to a release position. However, the housing portions 22, 24 can be movably connected together in various ways. Embodiments of the present inventive concept are not limited to the illustrated hinged configuration. Moreover, the housing 20′ can be manufactured as a single part with a hinge incorporated between the two portions 22, 24. A spring-like feature can be added to urge the housing portions 22, 24 to an open position when the locking pin 46 is moved to a release position.

Each housing portion 22, 24 includes a recessed portion 30 in each end, as illustrated in FIG. 7. When the housing portions 22, 24 are in the closed position, these recessed portions 30 create respective openings 32 in opposite ends of the housing 20′ through which catheters C can extend, as described above and illustrated in FIG. 2B. However, embodiments of the present inventive concept are not limited to the illustrated size, configuration, number or location of the openings in the housing 20′. The housing portions 22, 24 may be formed from various lightweight polymeric materials and are intended to be discarded after use by a single patient or if soiled during use by a patient.

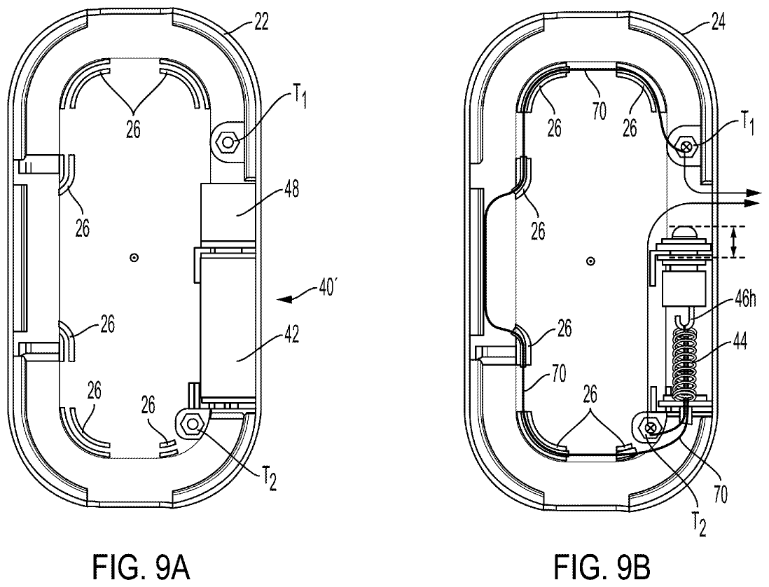

The locking mechanism 40′ is configured to secure the first and second housing portions 22, 24 in the closed position. In some embodiments, the locking mechanism is removably secured to the housing 20′ and is intended to be removed therefrom, sterilized and reused multiple times in other housings 20′. In the illustrated embodiment, housing portion 24 includes a cradle or receptacle 34 within which the locking mechanism can be removably secured, such as via a snap fit, etc. The illustrated locking mechanism 40′ includes a housing 42 containing a spring 44 and a locking pin 46, as illustrated in FIGS. 7 and 9B. The spring 44 is configured to urge the locking pin 46 outwardly from the housing 42 to an extended locking position. When the first and second housing portions 22, 24 are closed and the locking pin 46 is in the extended locking position, the locking pin 46 is inserted within a receiving portion 48 of the first housing portion 22, as illustrated in FIGS. 9A and 5B.

The locking mechanism 40′ includes a shape memory alloy (SMA) element 70 that is configured to be move the locking pin 46 from the extended locking position to a retracted release position. In the illustrated embodiment, the SMA element 70 is a wire that is connected at one end to a first electrical terminal T1 in the second housing portion 24. The SMA wire 70 extends around a path in the housing portion 24 and around a portion of the locking pin 46. The other end of the SMA wire 70 is secured to a second electrical terminal T2 in the housing portion 24. In the illustrated embodiment, the locking pin 46 includes a hook portion 46h (FIG. 9B) around which the SMA wire 70 extends. However, embodiments of the present inventive concept are not limited to the illustrated configuration. The SMA wire 70 may be connected to the locking pin 46 in a variety of ways without limitation.

In the illustrated configuration, a plurality of raised elements 26 are formed in the second housing portion 24 to create a path or channel for the SMA wire 70, as shown in FIGS. 7 and 9B. However, embodiments of the present inventive concept are not limited to the illustrated channel configuration. The SMA wire may extend from the first electrical terminal T1, around a portion of the locking pin 46, and then to the second electrical terminal T2 in a variety of paths and configurations without limitation.

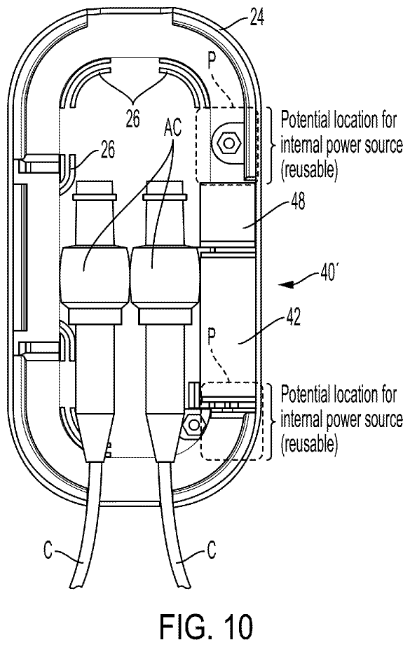

The first and second electrical terminals T1, T2 are connected to a power source P (FIG. 10), such as one or more batteries, via first and second wires P1, P2. When electric current is applied to the SMA wire 70 via the power source P, the SMA wire 70 is heated, which causes the SMA wire 70 to shorten its length and, thereby, pull the locking pin 46 from the extended locking position to a retracted release position. When the electrical current is no longer applied to the SMA wire 70, the spring 44 provides a restoring force to the SMA wire 70 to return the SMA wire 70 to its previous, pre-actuated length. In addition, the spring 44 is configured to urge the locking pin 46 to its extended locking position. An exemplary SMA material from which the SMA wire 70 is formed is a nickel titanium alloy, such as nitinol. The SMA wire 70 can have many different shapes/configurations which affects its pull distance. For example, a longer SMA wire 70 can pull the locking pin 46 a greater distance than a shorter SMA wire 70. Moreover, the SMA wire 70 need not be a wire. In some embodiments, it can have a knitted configuration, it can be a spring, etc.

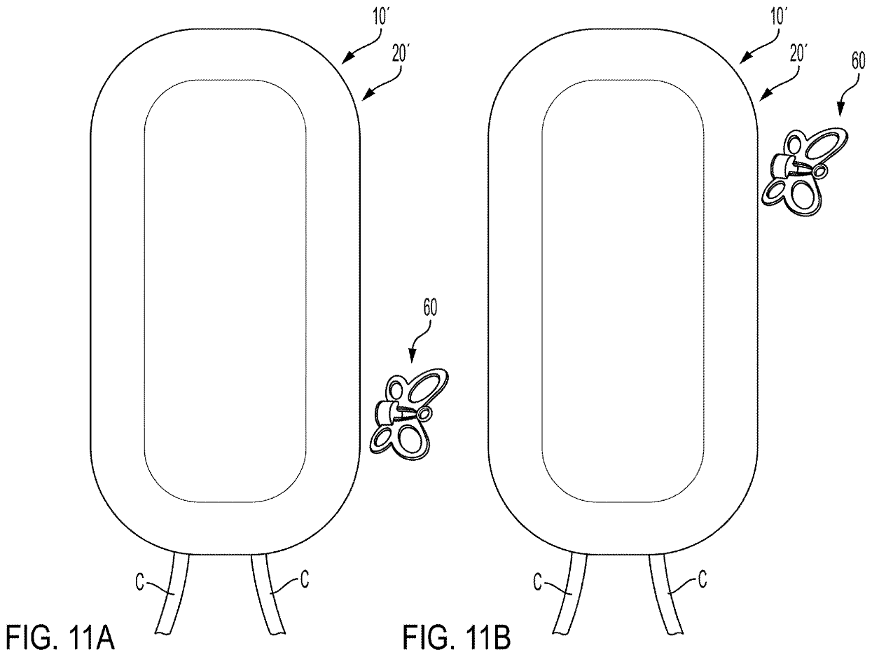

In operation, when the housing portions 22, 24 are moved to the closed position to encase the connection(s) AC of one or more catheters C therewithin, the activation key 60 is positioned adjacent to the housing 20′ (and need not make contact with the housing 20′) at a location of the power source P, as illustrated in FIGS. 11A-11B. This activates the power source P to supply electric current to the SMA wire 70, which heats the SMA wire 70 and causes the SMA wire to shorten its length and, thereby, pull the locking pin 46 from the extended locking position to a retracted release position. With the locking pin 46 in a retracted release position, the housing portions 22, 24 can be closed completely. The activation key 60 is then removed from the vicinity of the power source P and the SMA wire 70 quickly cools and returns to its previous length which allows the spring 44 to urge the locking pin 46 to the extended locking position so that the locking pin 46 is inserted within the receiving portion 48. To open the housing 20′ when in the closed position and the locking pin 46 is in the extended locking position, the activation key 60 is placed adjacent to the housing 20′ at the location of the power source P to activate the power source P to supply electric current to the SMA wire 70. The electric current heats the SMA wire 70 and causes it to shorten its length and thereby pull the locking pin 46 to a retracted release position, thereby allowing the housing portions 22, 24 to open. FIGS. 11A-11B illustrate positioning the activation key adjacent the respective locations of the power source P illustrated in FIG. 10 to cause the SMA wire to shorten its length and, thereby, pull the locking pin 46 from the extended locking position to a retracted release position.

In some embodiments, the activation key 60 communicates with a microcontroller that is associated with a circuit associated with the SMA wire 70. The communication may be via a unique signal that is accepted by the microcontroller, and then causes the microcontroller to close the circuit long enough to send current from the power source P (e.g., a battery in the housing 20′) to the SMA wire 70, thereby actuating it, and causing the locking pin 46 to move and the lock to open. The activation key 60 may contain a transmitter or a transceiver. The microcontroller associated with the SMA circuit may contain a transmitter, receiver, or transceiver.

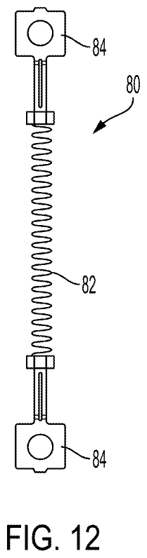

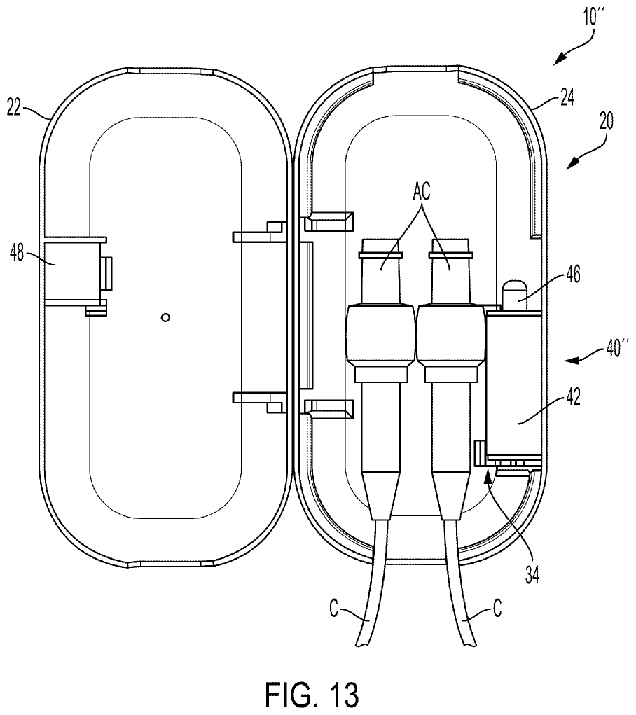

According to other embodiments of the present invention, and as described with respect to FIGS. 12, 13, 14A-14B and 15, an SMA spring 80 may be utilized to move the locking pin 46 of a locking mechanism 40″ to a retracted release position. FIG. 12 illustrates an SMA spring 80 according to some embodiments of the present inventive concept. The illustrated SMA spring 80 includes a coiled spring 82 of SMA material with connectors 84 at each end thereof.

The housing 20 within which the locking mechanism 40″ is located is similar to the housing 20 described above with respect to FIGS. 1, 2A-2B, 3, 4A-4B, 5A-5B and 6 and includes first and second portions 22, 24 configured to be movably connected to one another. The locking mechanism 40″ is configured to secure the first and second housing portions 22, 24 in the closed position. In some embodiments, the locking mechanism 40″ is removably secured to the housing 20 and is intended to be removed therefrom, sterilized, and reused multiple times in other housings 20. In the illustrated embodiment, housing portion 24 includes a cradle or receptacle 34 within which the locking mechanism can be removably secured, such as via a snap fit, etc.

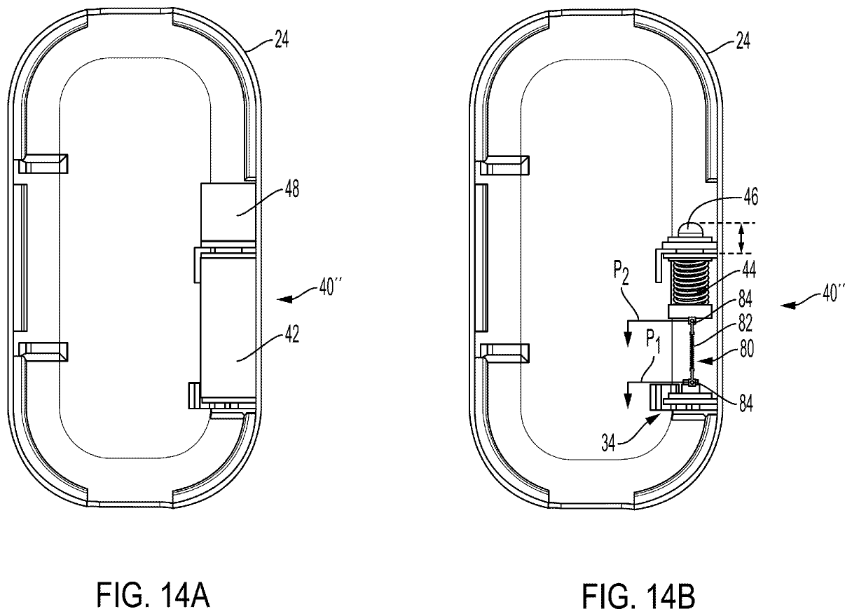

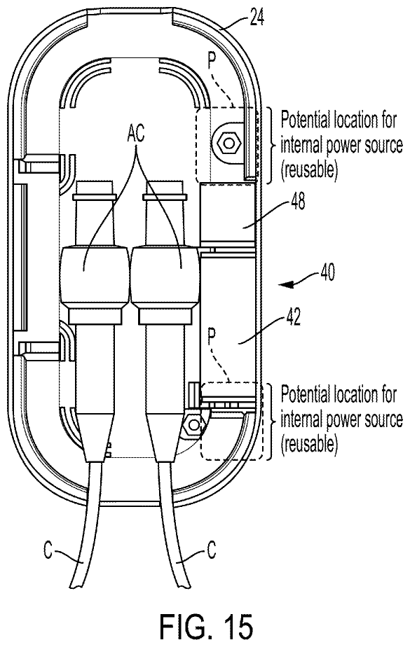

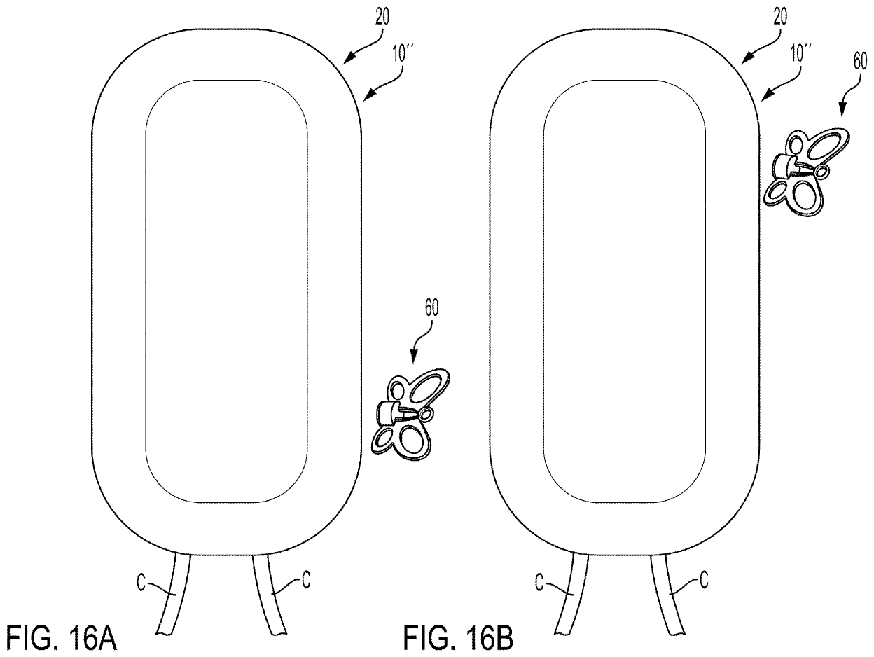

Referring to FIG. 14B, one connector 84 is attached to the locking pin 46 and the other connector 84 is connected to the housing 42 in which the SMA spring 80 is located. The connectors 84 are connected to a power source P (FIG. 15) via first and second wires P1, P2. When electric current is applied to the SMA spring 80 via the power source P, the coiled spring of SMA material 82 is heated, which causes the coiled spring 82 to shorten its length and, thereby, pull the locking pin 46 from the extended locking position to a retracted release position. The illustrated locking mechanism also includes a spring 44 that is configured to urge the locking pin 46 outwardly from the housing 42 to an extended locking position. In addition, the spring 44 is configured to restore the SMA spring 82 to it elongated configuration after activation. Thus, when the electric current is no longer applied to the SMA spring 80, the spring 44 restores the SMA spring 82 to its elongated configuration, thereby allowing it to be activated again, and the spring 44 also urges the locking pin 46 to its extended locking position. An exemplary SMA material from which the coiled spring 82 is formed is a nickel titanium alloy, such as nitinol. The SMA spring 80 can have many different shapes/configurations which affects its pull distance. For example, a longer SMA spring 80 can pull the locking pin 46 a greater distance than a shorter SMA spring 80. FIGS. 16A-16B illustrate positioning the activation key 60 adjacent the respective locations of the power source P illustrated in FIG. 15 to activate the power source P.

It will be understood that, although the terms first, second, etc., may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

The terms “catheter”, “catheter line”, “central venous line”, “blood vessel line”, and “vessel line” are used herein interchangeably herein and in a manner that is consistent with how one of ordinary skill in the art of the invention would understand these terms. A peripherally inserted central catheter (PICC) line, midline catheter line, and other non-central catheter lines are some examples of a “blood vessel line” or a “vessel line” that are commonly used to gain intravenous access to the blood stream for long term medical needs. In some embodiments, the enclosure apparatuses, systems, and methods described and illustrated herein may be configured with an exemplary PICC, midline catheter, or other non-central catheter line, although the enclosure apparatuses, systems, and methods may be configured to be used with other numbers, kinds, shapes, sizes, etc., of “catheters”, “catheter line(s)”, “central venous line”, “blood vessel line(s)”, or “vessel line(s)”.

It will be understood that when an element is referred to as being “on” another element, it can be directly on the other element or intervening elements may also be present. In contrast, when an element is referred to as being “directly on” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between”, “adjacent” versus “directly adjacent”, etc.).

Relative terms such as “below” or “above” or “upper” or “lower” or “horizontal” or “vertical” may be used herein to describe a relationship of one element, layer or region to another element, layer or region as illustrated in the figures. It will be understood that these terms are intended to encompass different orientations of the device in addition to the orientation depicted in the figures.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising,” “includes” and/or “including” when used herein, specify the presence of stated features, operations, elements, and/or components, but do not preclude the presence or addition of one or more other features, operations, elements, components, and/or groups thereof.

Aspects and elements of all of the embodiments disclosed above can be combined in any way and/or combination with aspects or elements of other embodiments to provide a plurality of additional embodiments.

1. A tamper-deterrent device for medical catheters, the device comprising:
a housing comprising first and second portions movably connected to one another and movable relative to one another between open and closed positions, wherein the first and second housing portions are configured to enclose a connection between at least two catheters when in the closed position; and
a locking mechanism configured to secure the first and second housing portions in the closed position, wherein the locking mechanism comprises a magnetically-operated locking pin configured to be moved by a contactless magnetic key from a locking position to a release position.

2. The device of claim 1, wherein the locking mechanism further comprises a spring configured to urge the locking pin to the locking position, and wherein the locking mechanism is located within the housing.

3. (canceled)

4. The device of claim 1, wherein the locking mechanism is removably secured to one of the first and second housing portions, and wherein the locking pin is configured to engage a portion of the other one of the first and second housing portions when in the locking position.

5. (canceled)

6. The device of claim 1, wherein the first and second housing portions are disposable.

7. The device of claim 1, wherein the housing comprises openings for the at least two catheters to extend therethrough, and wherein the openings are at opposite ends of the housing.

8. (canceled)

9. The device of claim 1, wherein the first and second housing portions are hingedly connected, and wherein the connection between the at least two catheters comprises access claves.

10. (canceled)

11. A tamper-deterrent device for medical catheters, the device comprising:
a housing comprising first and second portions movably connected to one another and movable relative to one another between open and closed positions, wherein the first and second housing portions are configured to enclose a connection between at least two catheters when in the closed position; and
a locking mechanism configured to secure the first and second housing portions in the closed position, wherein the locking mechanism comprises a locking pin configured to be moved from a locking position to a release position by a shape memory alloy (SMA) element when electric current is applied to the SMA element.

12. The device of claim 11, further comprising a power source located within the housing, wherein the power source is configured to provide the electric current to the SMA element in response to activation by a contactless key.

13. (canceled)

14. The device of claim 11, wherein the SMA element comprises a wire.

15. The device of claim 11, wherein the SMA element comprises nitinol.

16. The device of claim 11, wherein the locking mechanism further comprises a spring configured to urge the locking pin to the locking position, and wherein the locking mechanism is located within the housing.

17. (canceled)

18. The device of claim 11, wherein the locking mechanism is removably secured to one of the first and second housing portions, and wherein the locking pin is configured to engage a portion of the other one of the first and second housing portions when in the locking position.

19-20. (canceled)

21. The device of claim 11, wherein the housing comprises openings for the at least two catheters to extend therethrough, and wherein the openings are at opposite ends of the housing.

22. (canceled)

23. The device of claim 11, wherein the first and second housing portions are hingedly connected, and wherein the connection between the at least two catheters comprises access claves.

24. (canceled)

25. A tamper-deterrent device for medical catheters, the device comprising:
a housing comprising first and second portions movably connected to one another and movable relative to one another between open and closed positions, wherein the first and second housing portions are configured to enclose a connection between at least two catheters when in the closed position; and
a locking mechanism configured to secure the first and second housing portions in the closed position, wherein the locking mechanism comprises a locking pin and a shape memory alloy (SMA) spring, wherein the SMA spring is configured to move the locking pin from the locking position to a release position when electric current is applied to the SMA spring.

26. The device of claim 25, further comprising a power source located within the housing, wherein the power source is configured to provide the electric current to the SMA spring in response to activation by a contactless key.

27. (canceled)

28. The device of claim 25, wherein the SMA spring comprises nitinol, and wherein the locking mechanism is located within the housing.

29. (canceled)

30. The device of claim 25, wherein the locking mechanism is removably secured to one of the first and second housing portions, and wherein the locking pin is configured to engage a portion of the other one of the first and second housing portions when in the locking position.

31-32. (canceled)

33. The device of claim 25, wherein the housing comprises openings for the at least two catheters to extend therethrough, and wherein the openings are at opposite ends of the housing.

34. (canceled)

35. The device of claim 25, wherein the first and second housing portions are hingedly connected, and wherein the connection between the at least two catheters comprises access claves.

36. (canceled)
